(19) United States
(12) Patent Application Publication
(10) Pub. No.: US 2014/0080722 A1
(43) Pub. Date: Mar. 20, 2014

(54) METHOD OF SNP DETECTION BY USING DASH TECHNIQUE IN BEAD-BASED MICROFLUIDICS

(71) Applicants: Pei-Chun KAO, Taipei City (TW); Shih-Throng DING, Taipei City (TW); En-Chung LIN, Taipei City (TW); Yen-Wen LU, Taipei City (TW)

(72) Inventors: Pei-Chun KAO, Taipei City (TW); Shih-Throng DING, Taipei City (TW); En-Chung LIN, Taipei City (TW); Yen-Wen LU, Taipei City (TW)

(73) Assignee: NATIONAL TAIWAN UNIVERSITY, Taipei City (TW)

(21) Appl. No.: 13/618,671

(22) Filed: Sep. 14, 2012

(51) Int. Cl. C40B 30/04; G01N 21/64; C12Q 1/68

(52) U.S. Cl. 506/9; 435/6.11

(57) ABSTRACT

The present invention provides a method of SNP detection by using DASH technique in bead-based microfluidics comprising following steps: (a) immobilizing a target single-strand DNA onto a microbead; (b) hybridizing the target single-strand DNA with an allele-specific probe; (c) intercalating a dye into a target-probe duplex region; (d) delivering the microbead into a microchannel; (e) heating the microbead to denature a hybridized DNA obtained from the step (c); (f) monitoring a fluorescence intensity of the hybridized DNA during the step (e) to obtain a melting curve; and (g) determining the SNP by a melting curve analysis method. Also, the present invention offers a rapid genotyping detection scheme with minimal amount of the reagents by confining the microbeads into designed fluidic traps and performing melting curve analysis controlled by a temperature control platform. The trapping mechanism was validated and optimized.

### BACKGROUND OF THE INVENTION

1. Field of the Invention

The present invention relates to a method of SNP (Single-nucleotide polymorphism) detection by using DASH (Dynamic Allele-Specific Hybridization) technique. More particularly, the present invention relates to a method of SNP detection by using DASH technique in bead-based microfluidics.

2. Description of Related Art

Single nucleotide polymorphisms (SNPs) are one of the most common types in genetic variations, estimated to occur at 1 out of every 1,000 bases in the human genome, which means more than 10 million points of SNPs occurring across the human genome. SNPs are important markers that link sequence variations to phenotypic changes; such researches are expected to advance the understanding of human physiology and to elucidate the molecular bases of diseases. To date, a great deal of work has been devoted to developing accurate, rapid, and cost-effective technologies for SNP genotyping. The genotyping procedures typically involve the amplification of allele-specific products for SNP of interest, followed by the genotype detection techniques, such as enzymatic ligation, enzymatic cleavage, primer extension, split DNA enzymes G-quadruplex, sequencing, pyrosequencing, and mass spectroscopy. All of these techniques utilize enzymes, molecular beacon, or fluorescent dyes to label the DNA probes, leading to the requirement of high reagent cost or complicate procedures.

On the other hand, the dynamic allele-specific hybridization (DASH) technique has drawn great attention in SNP genotyping since it doesn't require the complex and expensive modification procedures on enzymes or fluorescent molecules. A conventional DASH procedure is described as follows. A target sequence is amplified by PCR in which one primer is biotinylated. The biotinylated product strand is bound to a streptavidin-coated microtiter plate well, and the non-biotinylated strand is rinsed away with alkali. An oligonucleotide probe, specific for one allele, is to hybridized to the target at low temperature. This forms a duplex DNA region that interacts with a double strand-specific intercalating dye. Upon excitation, the dye emits fluorescence proportional to the amount of double stranded DNA (probe-target duplex) present. The sample is then steadily heated while fluorescence is continually monitored. A rapid fall in fluorescence indicates the denaturing (or “melting”) temperature of the probe-target duplex. When performed under appropriate buffer and dye conditions, a single-base mismatch between the probe and the target results in a dramatic lowering of melting temperature (Tₘ) that can be easily detected.

In recent years, miniaturized devices, for instance microfluidic or lab-on-a-chip devices, have brought many advantages over their analogues at the macroscale, including portability, reduced sample consumption, rapid reaction times, and high throughput. Microfluidic devices with trapping mechanisms has been furthermore demonstrated to create a controlled microenvironment containing cells, particles and microbeads for monitoring and studying various dynamic and physiological activities. The microbeads can serve as a vehicle to immobilize the target biomolecules, carry the biomolecules for a series of reactions, and be trapped at desired position for further monitoring. Bead-based microfluidic devices thus can significantly simplified the tedious and labor-intensive washing procedures of traditional DNA/RNA purification and double-stranded DNA isolation process. The microbeads not only provide a relatively higher surface-to-volume ratio for biomolecule immobilization, but also have the advantages of enhancing reaction kinetics and reducing background noise.

### SUMMARY OF THE INVENTION

A primary objective of the present invention is to provide a method of SNP detection by using DASH technique in bead-based microfluidics.

To achieve the foregoing objective, the present invention provides a method of SNP detection by using DASH technique in bead-based microfluidics comprising following steps: (a) immobilizing a target single-strand DNA onto a microbead; (b) hybridizing the target single-strand DNA with an allele-specific probe; (c) intercalating a dye into a target-probe duplex region; (d) delivering the microbead into a microchannel; (e) heating the microbead to denature a hybridized DNA obtained from the step (c); (f) monitoring a fluorescence intensity of the hybridized DNA during the step (e) to obtain a melting curve; and (g) determining the SNP by a melting curve analysis method.

Preferably, before the step (a), the target single-strand DNA may be amplified by PCR.

In a preferred embodiment of the present invention, after the target single-strand DNA is amplified by PCR, the target single-strand DNA may be biotinlayted.

Preferably, before the step (a), the microbead may be coated with streptavidin.

Preferably, after the step (d), the microbead may be confined by a trap.

In a preferred embodiment of the present invention, the single trap may comprise the single microbead.

In a preferred embodiment of the present invention, the fluorescence intensity may be monitored by a CCD camera.

In a preferred embodiment of the present invention, the dye may bean intercalating dye.

Preferably, the intercalating dye may comprise SYBR Green I, EtBr or EVE Green.

In an aspect of the present invention, each of the microbead may be immobilized with the one allele-specific probe.

In another aspect of the present invention, each of the microbead may be immobilized with the plurality of allele-specific probes identifying different SNP types.

In a preferred embodiment of the present invention, a temperature of the step (e) may have a range from 55° C. to 95° C.

This summary is not an extensive overview of the disclosure and it does not identify key/critical elements of the present invention or delineate the scope of the present invention. Its sole purpose is to present some concepts disclosed herein in a simplified form as a prelude to the more detailed description that is presented later.

Many of the attendant features and advantages of the present invention will becomes better understood with reference to the following detailed description considered in connection with the accompanying drawings.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The present description will be better understood from the following detailed description read in light of the accompanying drawings, where:

FIGS. 1(a) to 1(e) are schematic flowchart of DASH technique in bead-based microfluidics for SNP genotyping;

FIGS. 2(a) to 2(b) depict the schematic illustration of the bead-based microfluidic: (a) The microbeads are injected into the microchannel and confined by the traps and (b) Two-dimensional COMSOL fluid velocity field simulation of bead-based microfluidic;

FIGS. 3(a) to 3(b) illustrate the conformation of the bead-based microfluidic chip: (a) micrograph from a microbeads trapping experiment, showing confinement of 20 μm diameter microbeads and (b) fluorescence micrographs of the target-probe duplex conjugated microbeads at 40° C., 65° C. and 80° C.;

FIG. 4 shows a photograph of an agarose gel electrophoresis of amplified DNA of the ATM-A gene region from two landrace sows run against a negative control: (M) DNA marker; (N) negative control; (1) asymmetric PCR product of wild type (CC genotype); (2) asymmetric PCR product of mutant type (TT genotype);

FIG. 5 illustrates a melting curve for two types of synthetic DNA samples of the ATM-A polymorphism;

FIG. 6(a) illustrates melting curves for three sow samples measuring melting curve by using the method of Rotor-Gene Q RT-PCR;

FIG. 6(b) illustrates melting curves for three sow samples measuring to melting curve by using the DASH technique in bead-based microfluidics of the present invention; and

FIG. 7 illustrates melting temperature for the samples of FC-363 (n=4) and FC-636 (n=4). (p=3×10⁻⁴)

### DESCRIPTION

The detailed description provided below in connection with the appended drawings is intended as a description of the present examples and is not intended to represent the only forms in which the present example may be constructed or utilized. The description sets forth the functions of the example and the sequence of steps for constructing and operating the example. However, the same or equivalent functions and sequences may be accomplished by different examples.

For convenience, certain terms employed in the specification, examples and appended claims are collected here. Unless defined otherwise, all technical and scientific terms used herein have the same meaning as commonly understood by one of the ordinary skill in the art to which this invention belongs.

The singular forms “a”, “and”, and “the” are used herein to include plural referents unless the context clearly dictates otherwise.

In the present invention, a genotyping system was established by integrating the DASH technique with a bead-based microfluidic device. The present invention not only preserved the flexible and accurate SNP detection scheme from the DASH technique, but it also possessed the advantage of having a minimal amount of the reagents. The microfluidic device confined the microbeads, thereby immobilizing the target DNA into designed fluidic traps, with the Melting Curve analysis then being conducted. Genotyping for both synthetic DNA and genomic DNA from Landrace sows on a SNP—ataxia-telangiectasia-mutated (ATM) gene—were discriminated via Melting Curve analysis. As the ATM gene in Landrace sows was recently found to play important roles in total number of piglets born, number born alive and average birth weight due to its differential expression between the morula and blastocyst stages, the present invention exhibited this bead-based SNP detection system with great potential being an effective approach to select useful biomarkers and to improve the reproductive traits in pigs.

To achieve the desired effect, the present invention offers a method of SNP detection by using DASH technique in bead-based microfluidics comprising following steps: (a) immobilizing a target single-strand DNA onto a microbead; (b) hybridizing the target single-strand DNA with an allele-specific probe; (c) intercalating a dye into a target-probe duplex region; (d) delivering the microbead into a microchannel; (e) heating the microbead to denature a hybridized DNA obtained from the step (c); (f) monitoring a fluorescence intensity of the hybridized DNA during the step (e) to obtain a melting curve; and (g) determining the SNP by a melting curve analysis method.

Before the step (a), the target single-strand DNA may be amplified by PCR. Furthermore, after the target single-strand DNA is amplified by PCR, the target single-strand DNA may be biotinlayted.

In addition, before the step (a), the microbead may be coated with streptavidin.

In particular, after the step (d), the microbead may be confined by a trap. In the present invention, the single trap may comprise the single microbead, and a diameter of the microbead may have a range from nanometer to micrometer. In other embodiment of the present invention, the single trap may comprise plurality microbeads.

In particular, a temperature of the step (e) may have a range from 55° C. to 95° C.

In a preferred embodiment of the present invention, the fluorescence intensity may be monitored by a CCD camera.

In a preferred embodiment of the present invention, the dye may be intercalating dye, such as SYBR Green I, EtBr, or EVE Green.

In an aspect of the present invention, each of the microbead may be immobilized with the one allele-specific probe.

In another aspect of the present invention, each of the microbead may be immobilized with the plurality of allele-specific probes identifying different SNP types.

The following descriptions are provided to elucidate certain aspects of the present invention and to aid those of skilled in the art in practicing this invention. These Examples are merely exemplary embodiments and in no way to be considered to limit the scope of the invention in any manner.

### Design and Working Principle

### Dynamic Allele-Specific Hybridization Method

In dynamic allele-specific hybridization (DASH) method of the present invention, an oligonucleotide probe, specific for one allele, is hybridized to the target. This forms a duplex DNA region that interacts with a double-stranded DNA (dsDNA) specific intercalating dye. Upon excitation, the dye emits fluorescence proportional to the amount of dsDNA. Therefore, when the sample is steadily heated, the dsDNA begins to denature and the amount of dsDNA decreases, leading to the decrease of the fluorescent intensity. When the temperature is close the melting temperature (Tₘ) of the probe-target duplex, the fluorescence intensity falls rapidly. An allele specific probe, which can form perfect dsDNA with wildtype single-stranded DNA (ssDNA) and one base pair mismatched with ssDNA containing SNP, is designed to separate the melting temperatures during the melting curve analysis. The present invention adapts this method and further conjugates the DNA sequences on microbeads to reduce the reagent use.

### Bead-Based Microfluidic Device for SNP Detection

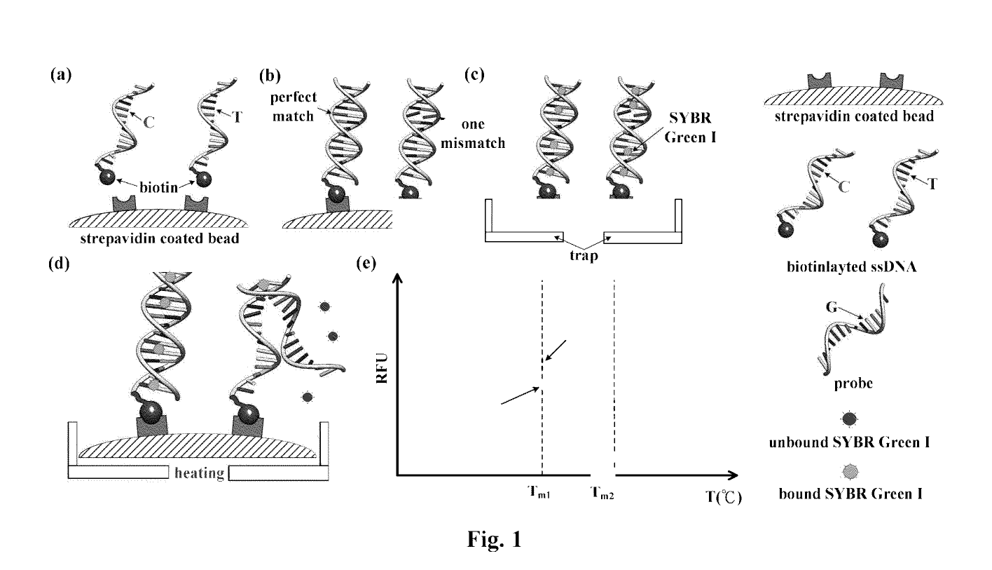

FIGS. 1(a) to 1(e) illustrate the proposed DASH technique on microbeads for SNP detection, and the steps are described as follows: (a) amplification of biotinlayted target ssDNA by PCR and immobilization onto streptavidin-coated bead; (b) hybridization of target ssDNA with allele-specific probe; (c) intercalation of SYBR Green I (i.e. Dye) into target-probe duplex region and confinement of bead in a trap; (d) heating and monitoring and fluorescence diminishing during temperature ramping to period; (e) illustration of melting curve analysis. (Tₘ₁: melting temperature of perfect-match sample, Tₘ₂: melting temperature of one-mismatch sample). In particular, Silica microbeads of 20 μm in diameter are coated with streptavidin and employed to bind with the biotinylated ssDNA. The DNA probe and intercalating dye are sequentially added to form the dye-intercalated probe-target conformation. After conjugated with the target-probe duplex, the microbeads are delivered into microchannels and confined by fluidic traps. The melting curve analysis is then conducted to in situ monitor the samples as the temperature increases during the DNA denaturation for the DASH technique. Due to the nucleotide mutation, one base mismatch between the target-probe duplex causes a lower Tₘ than the perfect match one; the SNP thus can be detected.

### Trapping Mechanism of Bead-Based Microfluidic

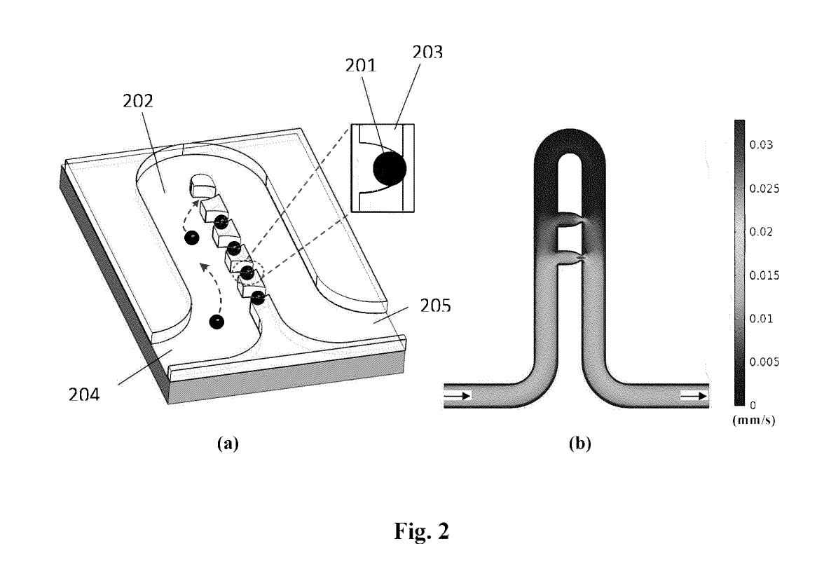

The bead-based microfluidic device consisted of the winding channels with arrays of fluidic traps, as shown in FIG. 2 (a). The microbeads 201 are injected into the microchannel 202 and confined by the traps 203. When the microbeads 201 flow inside the microchannels 202, microbeads 201 tend to go into and stay inside the traps 203 due to lower flow resistance. Fluid velocity field simulation was conducted by using the commercial finite element analysis software, COMSOL Multiphysics Version 4.0a (COMSOL, Inc., Burlington, Mass., USA). The model was based on the steady-state Nervier-Stokes' for an incompresible fluid. The parameters used in the simulation are as follows: 1000 kg/m³ for density, 1 mPa·s for dynamic viscosity. A uniform velocity of 0.01 m s⁻¹ was applied at the inlet 204 and a zero pressure boundary condition was applied at the outlet 205. No slip boundary conditions were set for the channel wells. As shown in the simplified Two-dimensional COMSOL fluid velocity field simulation of bead-based microfluidic in FIG. 2 (b), the flow velocity at the inlet was 0.01 mm s⁻¹, and increased to 0.033 mm s⁻¹ at the outlet of the first trap. The simulation result supported the hypothesis of the present invention that the flow velocity would increase at the outlet of the traps and cause a lower pressure to attract the microbeads.

### Material and Methods

A method of SNP detection by using DASH technique in bead-based microfluidics disclosed by the present invention will be described in further detail with reference to several aspects and examples below, which are not intended to limit the scope of the present invention.

### Microbead and Functionalization

Plain 20 μm polystyrene microbeads (Cat. 18329-5, Polysciences Inc., Warrington, Pa., USA) at 700 beads μL⁻¹ were used to validate the trapping efficiency of our microfluidic devices. For SNP detection, streptavidin-coated 20 μm diameter silica microbeads (Cat. 141048-05, Corpuscular Inc., Coldspring, N.Y., USA) at 250 beads μL⁻¹ were used to form the biological linker between the target sequence and microbeads (biotin-streptavidin).

### Microfluidic Device Fabrication and Design

The microfluidic device was fabricated via standard soft lithography process. Negative photoresist of SU-8 2025 (MicroChem, Newton, Mass., USA) was spin coated onto 4″ silicon wafer and patterned via photolithography. Silicone elastomer of polydimethylsiloxane (PDMS, from Sylgard 184, Dow Corning, Corning, N.Y., USA.) at 10:1 ratio was poured upon the SU-8 mold and cured at 150° C. After curing process, individual of microfluidic devices was first punched with biopsy punch (Kai medical, Seki City, Oyana, Japan.) to define the inlet and outlet of the microchannels. PDMS half curing method was utilized to covalent bond the devices. Briefly, PDMS was spin coated on well cleaned 1″×3″ glass slide and half curing at 60° C. for 30 min to form adhesive layer with a height of 20 μm. The cut microfluidic devices were then placed on the adhesive layer and hard cured at 150° C. for 15 min to complete the fabrication processes of bead-based microfluidic device. In addition, since the streptavidin-coated silica microbeads used in this study was 20 μm in diameter, the height and width of microchannel were designed at 30 μm and 80 μm respectively. Furthermore, the width of entry and exit for the trap were designed at 30 μm and 10 μm respectively, and the gap between each trap was 80 μm.

### Temperature Control Platform

A platform was developed to control the temperature of the microchannels for melting curve analysis. A 20 mm×32 mm of thin film polyimide heaters (Taiwan KLC, Taichung, Taiwan) was utilized to provide a uniform and stable heating source. A k-type negative temperature coefficient thermocouple (15.25Ω at 25° C.) was mounted on the backside of the microfluidic substrate to monitor the temperature. Data acquisition system (USB6210, National Instruments) and LabVIEW (National Instruments, Austin, Tex., USA) were used to acquire the resistance variance of the thermal couple, as well as to control the heater for thermal cycle. The performance of this temperature control platform was verified with the thermal imagers Ti50 (Fluke, Everett, Wash., USA).

### DNA Extraction, Amplification and SNP Discovery

The SNP discrimination point ATM-A lying on protein gene (Basic Local Alignment Search Tool: AY587061.1), which has proven to be a possible bio-marker associated with reproductive performance in Landrace sows, was chosen to demonstrate the validity and potential of our SNP detection system. For SNP discovery within the ATM gene, the total of three Landrace sows in Taiwan was used genomic DNA isolated from blood samples obtained from the anterior vena cava using a Puregene™ DNA Purification Kit based on the manufacturer's recommendations (Gentra System, Inc., MN, USA). The primer pairs for the ATM gene were designing by using a porcine nucleotide database (GenBank: AY587061). The translation start site of the ATM gene was present within the exon 3³⁰. To amplify the 5′-flanking region (upstream promoter and exon 1 to intron 2 region) sequence of the ATM gene by PCR, the primers were used, as listed in Table 1, led to the amplification of a 1,581-bp fragment. The purified PCR products were directly sequenced with these primers using an automated sequencer (ABI PRISM 3730 DNA Analyzer, Applied 50 Biosystems, Foster City, Calif., USA). The nucleotide sequences were aligned for the detection of SNPs using the program Lasergene (DNAstar, Madison, Wis., USA). For regulatory SNPs, binding motifs of transcription factors in the DNA fragments were estimated using MatInspector software (Genomatix, Munich, Germany).

### PCR Preparation

Two steps of PCR procedures, including symmetric PCR and asymmetric PCR, were performed to allow the target ssDNA with proper modifications to bind onto the microbeads. In the symmetric PCR process, genomic DNA was amplified to supply dsDNA containing target SNP point (ATM-A). In the asymmetric PCR process, the products from the first PCR were used as template, and the biotinlayted forward primers were applied to amplify target ssDNA. Moreover, in order to separate the dsDNA template and ssDNA target via agarose gel electrophoresis, primers were designed to produce different length of DNA sequences in each step, which were 91 bp and 73 mer, respectively. Meanwhile, the PCR conditions meanwhile were optimized to ensure sufficient and correct ssDNA were produced, after a series of tests on different concentrations of forward and reverse primers and the annealing temperature in both PCR steeps. The optimized PCR conditions are: 5 ng μL⁻¹ of genomic DNA, 200 μM dNTP mixture, 0.5 μM of Betaine, 1% of DMSO, 2.5 U Tag in a total reaction volume of 50 μL for the symmetric PCR. In addition, 0.2 μM of forward and reverse primers were used for the symmetric PCR to amplify the target genome region, and 0.5 μM of biotinlayted forward primer were used for the asymmetric PCR to amplify the single-strand DNA containing the ATM-A SNP point. The condition for the symmetric PCR: 94° C. for 5 min followed by 30 cycles of 94° C. for 20 sec, 55° C. for 30 sec, 72° C. for 20 sec. The condition for the asymmetric PCR: 94° C. for 5 min followed by 30 cycles of 94° C. for 20 sec, 52° C. for 30 sec, 72° C. for 20 sec. As shown in Table 1, all the oligonucleotides were purchased from Protect Technology Enterprise Co., Ltd. (Taipei, Taiwan) and used without further purification. The ssDNA probe was perfectly matched to the CC genotype sequence as well as to one-base-pair mismatched to the TT genotype sequence. This two-step PCR simplifies the tedious washing steps by removing the residual reagent and non-specific DNA sequences, promoting the target ssDNA binding onto the microbeads. This allowed the DASH technique to be conducted in the following procedures

TABLE 1

| Name | Sequence | |
|---|---|---|
| Primer-Forward (ATM) | 5′-CTCCCTCTCTACCGCGTCAACGCT-3′ | (SEQ. ID NO: 1) |
| Primer-Reverse (ATM) | 5′-CCCAGTAAGAGCATATGTTCAACAT-3′ | (SEQ. ID NO: 2) |
| Primer-1-Forward (ATM-A) | 5′-CTTACCCAATACCAGCCGGGCTA-3′ | (SEQ. ID NO: 3) |
| Primer-1-Reverse (ATM-A) | 5′-TTTTACCTGAGTCTCGTCTCTCA-3′ | (SEQ. ID N0: 4) |
| Primer-2-Forward (ATM-A) | 5′-Biotin-GGCTACGTCCGAGGG-3′ | (SEQ. ID NO: 5) |
| Probe-(C-type) | 5′-CCTGCGGCTTGGATCATGCTG-3′ | (SEQ. ID NO: 6) |

Names and sequences of the primers and probe used in ATM gene amplification, ATM-A gene amplification and melting curve analysis.
The boldface character represents the SNP position of ATM-A.

### Verification of Sample's Genotype

In order to verify the genotype of the samples used in this study, those samples have been previously genotyped by using commercial real-time PCR machine (MyiQ, Bio-Rad). Briefly, 10 μL of 2×SYBR Green I (Applied Biosystems, Beverly, Mass., USA) and 0.5 μL of probe (10 μM) were added to 10 μL of the product of asymmetric PCR. The melting curve analysis was then performed by holding at 94° C. for 3 min, 55° C. for 2 min and measuring the fluorescence signal for the temperature range of 60-90° C. The data for the melting curve analysis was then processed by using Bio-Rad iQ5 2.1 Standard Edition Optical System Software.

### Coupling of Synthetics DNA and PCR DNA to Microbeads

To immobilize the synthetics DNA, the reagent involved mixing of 0.4 μL of 10 μM 5′-biotinylated synthetic DNA, 0.4 μL of 10 μM probe, 10 μL of 2×SYBR Green I, 1 μL of streptavidin-coated microbead suspension (200 beads μL⁻¹), and 8.8 μL of ddH₂O. The mixture was then incubated at 60° C. for 30 min in a heat block (DB130-1, Firstek Ltd.) to enhance the binding efficiency of streptavidin-coated beads and biotinlayted target-probe duplexes. Meanwhile, to immobilize the DNA sequences from the animal samples, the 10 μL of asymmetric PCR products (ssDNA) and 10 μL of 2×SYBR Green 1 and 1 μL of 10 μM probe as well as 1 μL of streptavidin-coated microbead suspension (200 beads μL⁻¹) were mixing and incubated at 60° C. for 30 min in the heat block.

Genotyping with Melting Curve Analysis

Before the DNA samples were injected, the microchannel was washed with DI-water using the syringe pump at the flow rate of 60 μL min⁻¹. The mixing reagent was then directly injected into the microfluidic device at the flow rate of 30 μL min-1. Once the microbeads were completely confined by traps, DI-water was injected at the flow rate of 60 μL min⁻¹ to remove the residual reagents and separated the aggregated microbeads. The microfluidc device was then mounted on temperature control platform and assembled under an inverted fluorescence microscope (DMI3000, Leica). The microfluidic device was heated at 70° C. for 3 min to allow the affinity capture and avoid bubbles generating during the melting curve analysis. The microfluidic device was then heated from 60° C. to 90° C. The filters for excitation and emission were 425-475 nm and 600-660 nm according to the spectra of SYBR Green I. In order to avoid photo bleaching, a shutter was utilized during the temperature ramping period. Pictures were captured every 0.5° C. via a CCD camera and calibrated with software QCapture Pro (QImaging, Surrey, BC, Canada).

### Fluorescent Signal Quantification

Relative fluorescence intensity of the target-probe duplex on each 20 μm diameter microbead was quantified based on rom the fluorescent images by using image-processing software, ImageJ (NIH, Bethesda, Mass., USA). The microbeads, which showed higher initial fluorescent intensity and aggregation-free, were chosen as our candidates for DASH technique since they had more target DNA bound on the microbead surface. The fluorescence intensity was then represented by using a normalization function in the following equation:

$$Relative fluorescence intensity = \frac{{X}_{i} - {X}_{final}}{{X}_{initial}}$$

where Xᵢ was the fluorescent intensity of a microbead, X_{final} was the final fluorescent intensity of a microbead, and Xᵢₙᵢₜᵢₐₗ was the initial fluorescent intensity of a microbead. The melting curve profiles are normalized start from 100% and end at 0%. Besides, for statistical significance testing on our results, the p value corresponding to two homogeneous genotypes of ATM-A (CC and TT) was calculated via unpaired Student's t test. Differences with p values less than 0.05 were considered statistically significant.

### Results and Discussions

### Bead-Based Microfluidic Device

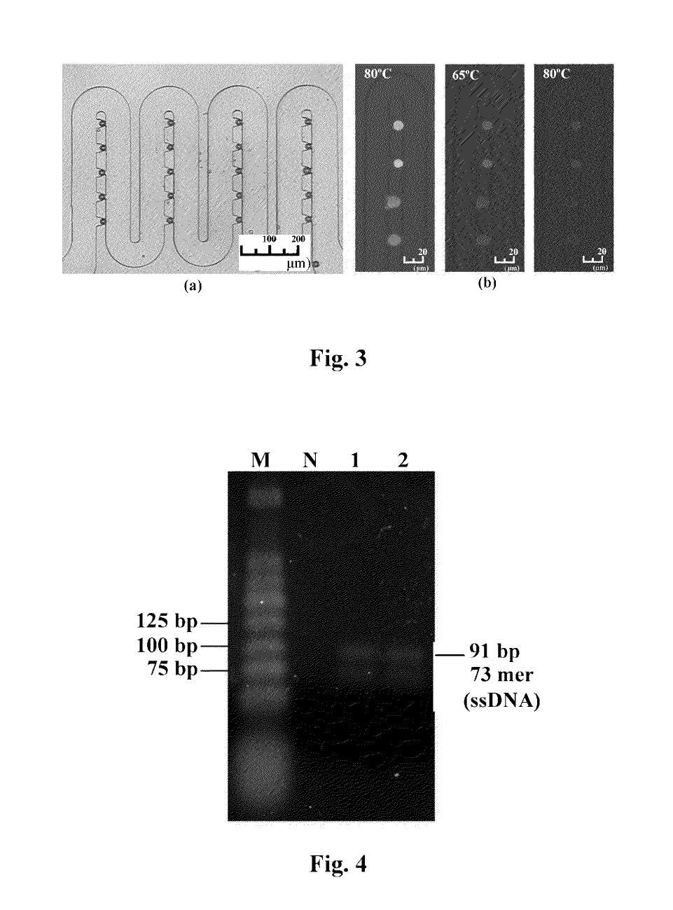

In the present invention, one fluidic trap containing one single microbead would be detected later in our bead-based microfluidic system. However, when multiple microbeads were captured by a single trap, aggregation of microbeads resulted. If aggregation continued, the flow would have been blocked and eventually obstructed by the microbeads. As a result, various combinations of geometric factors (such as the width and height of microchannel, the depth of the trap, as well as the width of inlet and outlet of each trap) were investigated to improve the trapping efficiency of the microfluidic device. The following combination was adapted in our device: the height and width of the microchannel in 30 μm and 80 μm, the depth of the trap in 30 μm, as well as the inlet and outlet for each trap in 30 μm and 10 μm. The bead-based microfluidic device was tested by injecting plain polystyrene microbeads of 20 μm. The experimental result in FIG. 3(a) validated that a single fluidic trap can contain one microbead with no aggregation. FIG. 3(b) shows the fluorescence micrographs of the target-probe duplex conjugated to the streptavidin microbeads at three different temperatures of 40° C., 65° C. and 80° C. during the melting curve analysis. The fluorescence signal on the microbeads decays as the temperature increased.

### SNP Detection Via a Bead-Based Microfluidic Device

To confirm the amplified DNA sequence, the PCR products of asymmetric PCR were visualized via electrophoresis on an 2% agarose gel stained with ethidium bromide and compared with the DNA marker (25/100 bp mixed DNA ladder, Bioneer). As shown in FIG. 4, the result brought out that the DNA fragments were 91 bp for as the DNA template and 73 mer for the biotinlayted target ssDNA sequence, as expected. As it revealed the amount of base-pairs in dsDNA, the intercalating dye of ethidium bromide could only exhibit in the self-folding regions for ssDNA, leading to a relatively weaker band. The 73-mer biotinlayted target ssDNA sequence, containing ATM-A SNP point, was also confirmed by the DNA sequencing instrument (Applied Biosystems 3730 DNA Analyzer).

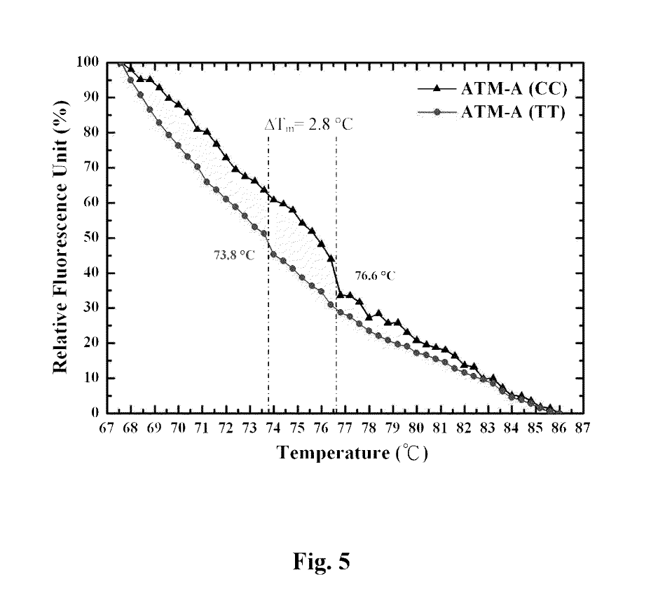

SNP genotyping analysis of each sample was performed using the bead-based microfluidic device by monitoring the fluorescence intensity of the target-probe duplex while the temperature ranged from 60° C. to 90° C. The fluorescence intensity data was then quantified and plotted. FIG. 5 shows the melting curves of both synthetic target DNA, perfect-match sample (CC), and one-base-pair-mismatch sample (TT). The genotypes were distinguished by observing the decreasing trend of each curve. The profile of the perfect-match sample (CC) had a lower decreasing rate due to the larger binding forces between the probe and target ssDNA. The maximum slope change or the minimum value of the first derivatives of the melting curve was determined as the melting temperature. The melting temperature difference (ΔTₘ) for two types of the synthetic samples was 2.8° C. In addition, only twenty microbeads were used in the present invention. This was relatively less than the reagent amount commonly-consumed in the traditional DASH technique

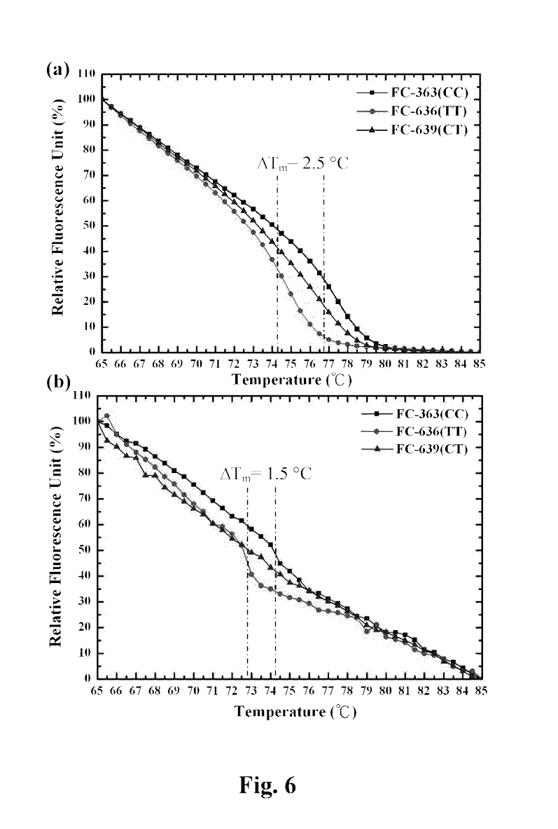

FIG. 6 shows the results of the Melting Curve analysis on three sow samples (FC-363 (CC), FC-636(TT), FC-639(CT)). All Melting Curve analysis results were conducted using the system of the present invention and confirmed by Rotor-Gene Q instrument. The curves were relatively smoother in Rotor-Gene Q system due to the amount of dsDNA and better temperature controller. Rotor-Gene Q utilized PCR tubes, which contained considerably more probe-target dsDNA than a single microbead. Furthermore, the resolution of temperature controller was 0.02° C. in the RT-PCR system and 0.2° C. in the present invention. This affected the curvature of the profiles. The background noise was lower in RT-PCR system than in the bead-based microfluidic. The melting temperature difference (ΔTₘ) for the CC and TT types of the samples was 2.5° C. when Rotor-Gene Q system was employed, and was 1.5° C. when the system of the present invention was used.

The heterozygous sample (CT) showed a combinative profile of both homozygous samples (CC and TT) as expected. The results of both the systems were consistent, which validated the reliability of SNP detection method of the present invention. It was worth mentioning that the peaks of each genotype shifted from 74.25° C. to 72.75° C. and 76.75° C. to 74.25° C. in the present invention while temperature differences were similar. One of the major reasons for this temperature shift was that the melting temperature depended on the heating rate. A higher heating rate usually caused a higher melting peak due to the shifting of melting temperatures. In comparison with the heating rate of 3° C. min⁻¹ from the commercial RT-PCR instrument, the present invention had a 2° C. min⁻¹ heating rate—which was relatively slower. Thus, the melting temperatures shifted to lower values.

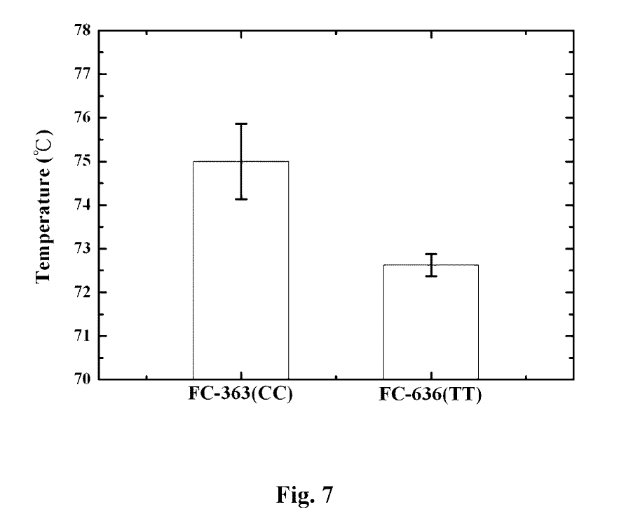

The results for the melting curves analysis are shown in FIG. 7. At least four microbeads with relative higher fluorescence signal were chosen and quantified to plot the melting curves in each experiment. The melting temperatures for each sample were determined when the derivative of each melting curves reached the negative maximum. The average value of the melting temperature from three independent experiments in the CC genotype sample FC-363 was 75±0.71° C., with the average value of the melting temperature for the TT genotype sample FC-636 being 72.63±0.25° C. The p value was 3×10⁻⁴, proving that the perfect-match sample (CC) could be statistically distinguished from the one-mismatch sample (TT).

The SNP detection results of the present invention were consistent with the results from Rotor-Gene Q instrument. To further confirm the validity of SNP detection for both systems using DASH method, independent SNP detection was conducted using BeadXpress (Illumina, Inc., San Diego, Calif., USA) with the GoldenGate genotyping assay performed at the Center of Biotechnology at National Taiwan University. All preceding SNP detection results are summarized in Table 2. The SNP genotyping results from the samples with three possible genotypes were successfully identified, validating the comparison of the results obtained from the Rotor-Gene Q, the system of the present invention, and the GoldenGate genotyping assay performed at the Center of Biotechnology, National Taiwan University, Taiwan.

TABLE 2

| | Genotyping Result | | |
|---|---|---|---|
| Landrace | Rotor-Gene | Bead-based | GoldenGate |
| sow | Q | microfluidic device | genotyping assay |
| FC-363 | CC | CC | CC |
| FC-636 | TT | TT | TT |
| FC-518 | CT | CT | CT |

Results for genotyping of the landrace sows acquired from the PCR amplification for the SNP ATM-A. The table lists the comparison of the results obtained from the Rotor-Gene Q, the developed bead-based microfluidic device, and the Golden gate genotyping assay performed at the Center of Biotechnology, National Taiwan University, Taiwan.

To sum up, a novel SNP genotyping system of the present invention is developed by conducting DASH technique on microbeads in microfluidic devices. The DNA duplexes were conjugated onto silica microbeads and the melting curve analysis was performed with our temperature control platform. Also, SNP detection on single microbead was achieved in 20 min. The genotyping results of ATM-A mutation were compared to the results obtained from commercial genotyping instrument, verifying the reliability of the developed system. Finally, the present invention can be further integrated with PCR capability to simplify the DNA amplification and isolation procedures. The volume reduction and rapid analysis that the present invention can provide the potential of being a cost-effective and high-throughput SNP detection method in genotyping applications.

In accordance with the present invention, the method of SNP detection by using DASH technique in bead-based microfluidics has the following advantages:

(1) SNP detection on single microbead was achieved in 20 min, less than the time the conventional DASH technique needs.

(2) Compared with conventional DASH technique, samples of the present invention can be assembled by the microbeads (solid support), such that not merely can the background interference be reduced but the sensitivity and the detection limit can also be increased.

(3) The plurality of microbeads with different size can be employed to deliver to microchannels, and the surface of those microbeads are immobilized the different probes, such that different SNP types can be identified, and high-throughput and multiplex SNP detection method in genotyping applications can be employed.

It will be understood that the above description of embodiments is given by way of example only and that various modifications may be made by those with ordinary skill in the art. The above specification, examples, and data provide a complete description of the present invention and use of exemplary embodiments of the invention. Although various embodiments of the invention have been described above with a certain degree of particularity, or with reference to one or more individual embodiments, those with ordinary skill in the art could make numerous alterations to the disclosed embodiments without departing from the spirit or scope of this invention.

1. A method of single nucleotide polymorphism (SNP) detection by using Dynamic Allele-Specific Hybridization technique in bead-based microfluidics comprising the following steps:
(a) immobilizing a target single-strand DNA onto a microbead;
(b) hybridizing the target single-strand DNA with an allele-specific probe;
(c) intercalating a dye into a target-probe duplex region;
(d) delivering the microbead into a microchannel;
(e) confining the microbead by a trap;
(f) heating a portion of the microchannel, which comprises the trap and the microbead, to denature a hybridized DNA obtained from the step (c);
(g) monitoring a fluorescence intensity of the hybridized DNA during the step (f) to obtain a melting curve; and
(h) determining the SNP by a melting curve analysis method.

2. The method of claim 1, wherein before the step (a), the target single-strand DNA is amplified by PCR.

3. The method of claim 2, wherein after the target single-strand DNA is amplified by PCR, the target single-strand DNA is biotinylated.

4. The method of claim 2, wherein before the step (a), the microbead is coated with streptavidin.

5. (canceled)

6. The method of claim 1, wherein a single trap comprises a single microbead.

7. The method of claim 1, wherein the fluorescence intensity is monitored by a CCD camera.

8. The method of claim 1, wherein the dye is an intercalating dye.

9. The method of claim 8, wherein the intercalating dye comprises SYBR Green I, EtBr or EVE Green.

10. The method of claim 1, wherein each microbead is immobilized with one allele-specific probe.

11. The method of claim 1, wherein each microbead is further immobilized with a plurality of allele-specific probes identifying different SNP types.

12. The method of claim 1, wherein a temperature of the step (f) ranges from 55° C. to 95° C.

13. The method of claim 1, wherein the trap prevents the microbead from moving.


SEQUENCE LISTING (6 sequences)

SEQ ID NO: 1 - DNA, 24 bp, Artificial Sequence
  Primer
ctccctctctaccgcgtcaacgct

SEQ ID NO: 2 - DNA, 25 bp, Artificial Sequence
  Primer
cccagtaagagcatatgttcaacat

SEQ ID NO: 3 - DNA, 23 bp, Artificial Sequence
  Primer
cttacccaataccagccgggcta

SEQ ID NO: 4 - DNA, 23 bp, Artificial Sequence
  Primer
ttttacctgagtctcgtctctca

SEQ ID NO: 5 - DNA, 15 bp, Artificial Sequence
  Primer
ggctacgtccgaggg

SEQ ID NO: 6 - DNA, 21 bp, Artificial Sequence
  Primer
cctgcggcttggatcatgctg